(19) United States
(12) Patent Application Publication
(10) Pub. No.: US 2015/0342538 A1
(43) Pub. Date: Dec. 3, 2015

(54) CUSTOM EARLY WARNING SCORING FOR MEDICAL DEVICE

(71) Applicant: Welch Allyn, Inc., Skaneateles Falls, NY (US)

(72) Inventors: Shawn C. St. Pierre, Syracuse, NY (US); Kristin A. Alisanski, Syracuse, NY (US); Frank LoMascolo, Auburn, NY (US); Catherine M. King, Ithaca, NY (US); Kathryn M. Coles, Syracuse, NY (US); Thomas A. Myers, Syracuse, NY (US)

(21) Appl. No.: 14/528,487

(22) Filed: Oct. 30, 2014

Related U.S. Application Data
(60) Provisional application No. 62/007,302, filed on Jun. 3, 2014.

(51) Int. Cl. A61B 5/00; A61B 5/1455; A61B 5/145; A61B 5/0205

(52) U.S. Cl. CPC A61B 5/7275; A61B 5/02055; A61B 5/14551; A61B 5/14535; A61B 5/742; A61B 5/746; A61B 5/7405; A61B 5/0022; A61B 5/01

(57) ABSTRACT

A method for providing an early warning system for a patient includes: allowing a caregiver to select among a plurality of early warning score protocols; allowing the caregiver to modify the selected early warning score protocol; automatically calculating an early warning score for the patient; and displaying the early warning score for the caregiver. A medical device includes at least one health care equipment module configured to measure a physiological parameter of a patient. The medical device is configured to allow the caregiver to select among a plurality of early warning score protocols. The early warning score protocols may be targeted to specific patients based on demographic information. The medical device also allows the caregiver to modify the selected early warning score protocol. The medical device also automatically calculates an early warning score for the patient using a physiological parameter captured by the at least one health care equipment module.

### CROSS REFERENCE TO RELATED APPLICATION

This application claims priority to U.S. Ser. No. 62/007,302, titled CUSTOM EARLY WARNING SCORING FOR MEDICAL DEVICE, filed on Jun. 3, 2014, the disclosure of which is hereby incorporated by reference in its entirety.

### BACKGROUND

Research studies indicate that 80% of patients typically exhibit at least one psychological warning sign up to 24 hours prior to their condition worsening. A research study in Australia performing a retrospective chart analysis reported similar results. The study reported that 77% of patients transferred from medical/surgical floors to the ICU had at least one vital sign indicator outside of normal range documented in their chart within 8 hours of their admission. Additionally, the study reported that these indicators go unnoticed for a variety of reasons, including variance to standard protocols, lack of staff communication, and lack of advanced clinical training/knowledge.

### SUMMARY

In one aspect, a method for providing an early warning system for a patient includes: allowing a caregiver to select among a plurality of early warning score protocols; allowing the caregiver to modify the selected early warning score protocol; automatically calculating, by a medical device, an early warning score for the patient; and displaying the early warning score for the caregiver.

In another aspect a method for defining an early warning score protocol for a patient, the method comprising: identifying a plurality of physiological parameters; defining a mapping from the plurality of physiological parameters to a plurality of component score; defining a calculation method for combining the plurality of component scores into an early warning score; specifying a plurality of ranges for the early warning score; and inputting instructions for at least one of the plurality of ranges.

In yet another aspect a medical device comprising: at least one health care equipment module configured to measure at least one physiological parameter of a patient; a display screen; a computing device, the computing device comprising: at least one processing device; and at least one computer readable data storage device storing instructions that, when executed by the at least one processing device, cause the medical device to: allow a caregiver to select among a plurality of early warning score protocols, wherein at least one of the early warning score protocols is targeted to specific patients based on demographic information; allow the caregiver to modify the selected early warning score protocol; automatically calculate an early warning score for the patient using a physiological parameter captured by the at least one health care equipment module; and display a user interface on the display screen, the user interface including the early warning score and instructions for the caregiver that are selected based on the early warning score.

### DESCRIPTION OF THE FIGURES

FIG. 1 shows an example system for maintaining medical devices.

FIG. 2 shows an example medical device of the system of FIG. 1.

FIG. 3 shows another view of the medical device of FIG. 2.

FIG. 4 shows another example medical device of the system of FIG. 1.

FIG. 5 shows another example medical device of the system of FIG. 1.

FIG. 6 shows an example user interface of the medical devices of FIG. 1.

FIG. 7 shows another example user interface of the medical devices of FIG. 1.

FIG. 8 shows another example user interface of the medical devices of FIG. 1.

FIG. 9 shows another example user interface of the medical devices of FIG. 1.

FIG. 10 shows another example user interface of the medical devices of FIG. 1.

FIG. 11 shows an example architecture of the medical devices of FIG. 1.

FIG. 12 shows an example process performed by some embodiments of the medical devices of FIG. 1 to define an early warning score protocol.

FIG. 13 shows an example process performed by some embodiments of the medical devices of FIG. 1 to modify and use an early warning score protocol.

FIG. 14 shows an example process performed by some embodiments of the medical devices of FIG. 1 to calculate and display an early warning score.

FIG. 15 shows example components of a medical device of the system of FIG. 1.

### DETAILED DESCRIPTION

The present disclosure relates to medical devices that are used to collect physiological data from patients.

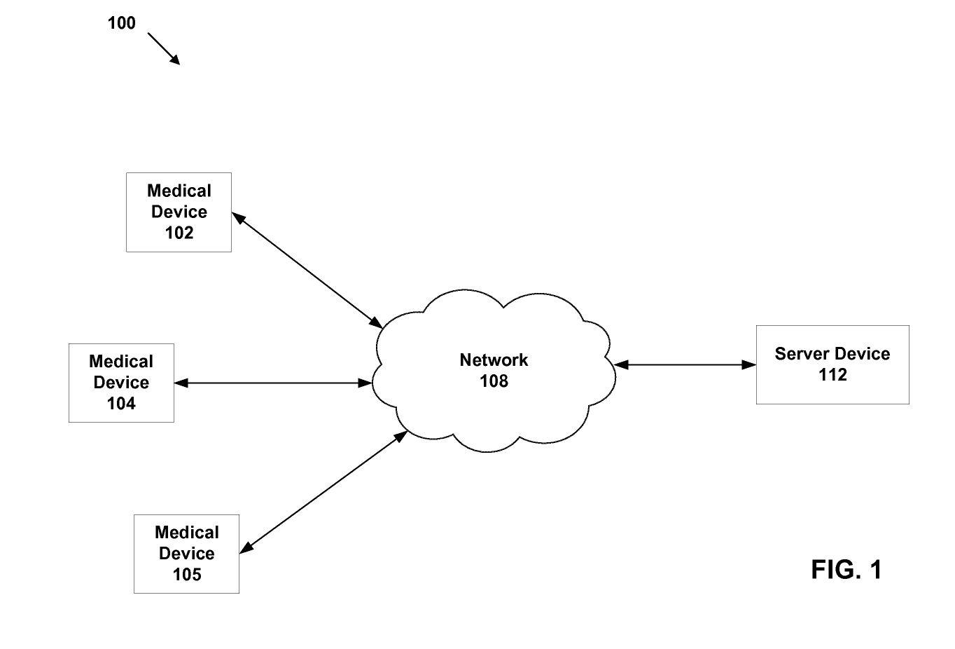

FIG. 1 is a block diagram illustrating an example system 100 for medical devices.

In this example, medical devices 102, 104, 105 are used to collect physiological data from patients. These medical devices can be located in a facility, such as a hospital or clinic. In one example, the devices 102, 104, 105 are located at the same facility. In another example, the devices are located at different facilities spread out geographically.

The medical devices 102, 104, 105 communicate with a network 108. In one example, the medical devices 102, 104, 105 and the network 108 are part of a CONNEX™ system from Welch Allyn of Skaneateles Falls, N.Y., although other systems can be used. In such an example, the medical devices communicate through known protocols, such as the Welch Allyn Communications Protocol (WACP). WACP uses a taxonomy as a mechanism to define information and messaging. Taxonomy can be defined as description, identification, and classification of a semantic model. Taxonomy as applied to a classification scheme may be extensible. Semantic class-based modeling utilizing taxonomy can minimize the complexity of data description management by limiting, categorizing, and logically grouping information management and operational functions into families that contain both static and dynamic elements.

The network 108 is an electronic communication network that facilitates communication between the medical devices 102, 104, 105. An electronic communication network is a set of computing devices and links between the computing devices. The computing devices in the network use the links to enable communication among the computing devices in the network. The network 108 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various embodiments, the network 108 includes various types of links. For example, the network 108 can include wired and/or wireless links. Furthermore, in various embodiments, the network 108 is implemented at various scales. For example, the network 108 can be implemented as one or more local area networks (LANs), metropolitan area networks, subnets, wide area networks (such as the Internet), or can be implemented at another scale.

In this example, the medical devices 102, 104, 105 and the network 108 are all part of the same network. In other words, the medical devices 102, 104, 105 and the network 108 communicate with one another over a LAN behind a wall safeguarding the devices from outside influences on the Internet, such as a firewall.

The medical devices 102, 104, 105 can provide various types of functionality, including measuring or monitoring patient physiological parameters. The medical devices 102, 104, 105 can include one or more physiological monitor devices configured to measure and possibly display representations of one or more physiological parameters of a patient. In addition, the medical devices 102, 104, 105 can include one or more desktop, laptop, or wall-mounted devices. In some embodiments, the medical devices 102, 104, 105 are configured to be used by a clinician to monitor the physiological parameters of multiple patients at one time. Such monitor devices are typically not wall mounted.

The medical devices 102, 104, 105 can communicate with each other through the network 108. In various embodiments, the medical devices 102, 104, 105 can communicate various types of data with each other through the network 108. For example, in embodiments where the medical devices 102, 104, 105 includes a set of physiological monitor devices and a monitor device, each of the physiological monitor devices can send data representing measurements of physiological parameters of patients to the monitor device. In this way, the medical devices 102, 104, 105 can display representations of physiological parameters to a clinician.

The medical devices 102, 104, 105 can send various types of data and can receive various types of data through the network 108. For example, in some embodiments, the medical devices 102, 104, 105 can send measurements of physiological parameters. In another example, the medical devices 102, 104, 105 can retrieve past measurements of physiological parameters of patients.

A server device 112 communicates through the network 108 with the medical devices 102, 104, 105. In this example, the server device 112 monitors the status of the medical devices 102, 104, 105 to determine various attributes of the medical devices 102, 104, 105, such as maintenance requirements and upgrade requirements.

In this example, the server device 112 is located “in the cloud.” In other words, the server device 112 is located outside of the internal network associated with the medical devices 102, 104, 105. Typically, the server device 112 does not communicate directly with the medical devices 102, 104, 105, but instead communicates with a central server located within the same network as the medical devices 102, 104, 105, such as the CONNEX™ system from Welch Allyn of Skaneateles Falls, N.Y. Intermediary servers in the CONNEX™ system, in turn, communicate with the medical devices 102, 104, 105. Other configurations are possible.

The medical devices 102, 104, 105 and the server device 112 are computing systems. As used herein, a computing system is a system of one or more computing devices. A computing device is a physical, tangible device that processes data. Example types of computing devices include personal computers, standalone server computers, blade server computers, mainframe computers, handheld computers, smart phones, special purpose computing devices, and other types of devices that process data.

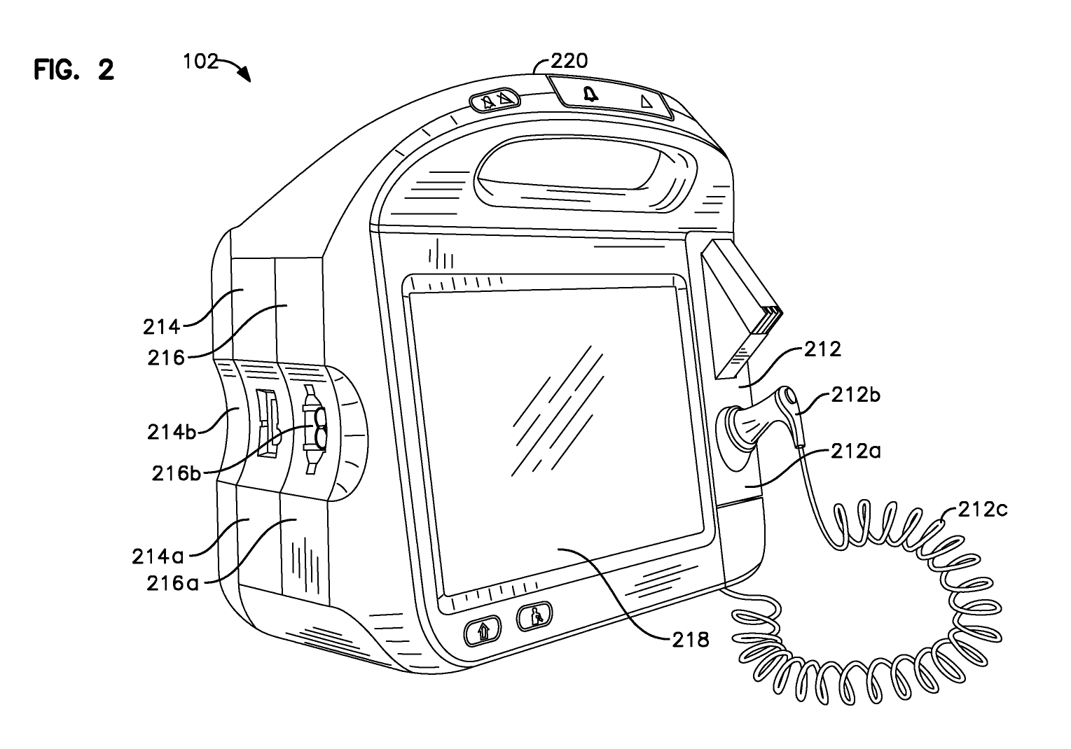

FIG. 2 illustrates one example of the medical device 102. The medical device 102 is portable. The medical device 102 includes multiple health care equipment (HCE) modules. Each of the HCE modules is configured to measure one or more physiological parameters of a health-care recipient, also referred to herein as a patient. Other embodiments can include more or fewer components than those shown in FIG. 2, or can include different components that accomplish the same or similar functions.

A temperature measurement module 212 is accessible from the front side of the medical device 102. A SpO2 module 214 and a non-invasive blood pressure (NIBP) module 216 are accessible from a left hand side of the medical device 102. An upper handle portion 220 enables the medical device 102 to be carried by hand.

A front side of the medical device 102 includes a display screen 218 and an outer surface of the temperature measurement module 212. The temperature measurement module 212 is designed to measure the body temperature of a patient. As used in this document, a “module” is a combination of a physical module structure which typically resides within the medical device 102 and optional peripheral components (not shown) that typically attach to and reside outside of the medical device 102.

The temperature measurement module 212 includes a front panel 212a. The front panel 212a has an outer surface that is accessible from the front side of the medical device 102. The front panel 212a provides access to a wall (not shown) storing a removable probe (not shown), also referred to as a temperature probe, that is attached to a probe handle 212b. The probe and its attached probe handle 212b are tethered to the temperature measurement module 212 via an insulated conductor 212c. The probe is designed to make physical contact with a patient in order to sense a body temperature of the patient.

A left hand side of the medical device 102 includes an outer surface of the SpO2 module 214 and an outer surface of the NIBP module 216. The SpO2 module 214 is a HCE module designed to measure oxygen content within the blood of a patient. The NIBP module 216 is a HCE module designed to measure blood pressure of a patient.

As shown, the SpO2 module 214 includes a front panel 214a. The front panel 214a includes an outer surface that is accessible from the left side of the medical device 102. The front panel 214a includes a connector 214b that enables a connection between one or more peripheral SpO2 components (not shown) and a portion of the SpO2 module 214 residing inside the medical device 102. The peripheral SpO2 components reside external to the medical device 102. The peripheral SpO2 components are configured to interoperate with the SpO2 module 214 when connected to the SpO2 module 214 via the connector 214b. In some embodiments, the peripheral SpO2 components include a clip that attaches to an appendage of a patient, such as a finger. The clip is designed to detect and measure a pulse and an oxygen content of blood flowing within the patient.

As shown, the NIBP module 216 includes a front panel 216a having an outer surface that is accessible from the left side of the medical device 102. The front panel 216a includes a connector 216b that enables a connection between one or more peripheral NIBP components (not shown) and a portion of the NIBP module 216 residing inside the medical device 102. The peripheral NIBP components reside external to the medical device 102. The peripheral NIBP components are configured to interoperate with the NIBP module 216 when connected to the NIBP module 216 via the connector 216b. In some embodiments, the peripheral NIBP components include an inflatable cuff that attaches to an appendage of a patient, such as an upper arm of the patient. The inflatable cuff is designed to measure the systolic and diastolic blood pressure of the patient, the mean arterial pressure (MAP) of the patient, and the pulse rate of blood flowing within the patient.

The medical device 102 is able to operate within one or more workflows (or profiles). A workflow is a series of one or more tasks that a user of the medical device 102 performs. When the medical device 102 operates within a workflow, the medical device 102 provides functionality suitable for assisting the user in performing the workflow. When the medical device 102 operates within different workflows, the medical device 102 provides different functionality.

When the medical device 102 is manufactured, the medical device 102 is configured to be able to operate within one or more workflows. After the medical device 102 is manufactured, the medical device 102 can be reconfigured to operate within one or more additional workflows. In this way, a user can adapt the medical device 102 for use in different workflows as needed.

In various embodiments, the medical device 102 operates within various workflows. For example, in some embodiments, the medical device 102 can operate within a monitoring workflow or a non-monitoring workflow. Example types of non-monitoring workflows include, but are not limited to, a spot check workflow, an office workflow, and a triage workflow. A non-limiting example of a monitoring workflow is an intervals workflow.

In example embodiments, the names for the workflows can be defined by the user. For example, the user can rename a “triage workflow” as “ED 3 North” or any other nomenclature as desired to provide more context to the user.

When the medical device 102 is operating within the monitoring workflow, the medical device 102 obtains a series of measurements of one or more physiological parameters of a single monitored patient over a period of time. In addition, the medical device 102 displays, on the display screen 218, a monitoring workflow home screen. The monitoring workflow home screen contains a representation of a physiological parameter of the monitored patient. The representation is based on at least one measurement in the series of measurements. A representation of a physiological parameter is a visible image conveying information about the physiological parameter.

For example, when the medical device 102 is operating within the monitoring workflow, the medical device 102 can obtain a blood pressure measurement of a single patient once every ten minutes for six hours. In this example, the medical device 102 displays a monitoring workflow home screen that contains a representation of the patient's blood pressure based on a most recent one of the blood pressure measurements. In this way, a user of the medical device 102 can monitor the status of the patient.

When the medical device 102 is operating within a non-monitoring workflow, the medical device 102 obtains a measurement of one or more physiological parameters from each patient in a series of patients. In addition, the medical device 102 displays a non-monitoring workflow home screen on the display screen 218. The non-monitoring workflow home screen contains a representation of the physiological parameter of a given patient in the series of patients. The representation is based on the measurement of the physiological parameter of the given patient.

In one example, when the medical device 102 is operating within a spot check workflow, the medical device 102 obtains blood pressure measurements from a series of previously-identified patients. In this other example, the medical device 102 displays a spot check workflow home screen containing a blood pressure measurement of a given patient in the series of previously-identified patients. In this way, a user of the medical device 102 can perform spot checks on the blood pressures of patients who have already been admitted to a hospital.

As used in this document, a patient is a previously identified patient when the medical device 102 stores information regarding the identity of the patient. In another example, when the medical device 102 is operating within a triage workflow, the medical device 102 can obtain a single blood pressure measurement from each patient in a series of unidentified patients as the patients arrive at a hospital. In this example, the medical device 102 displays a triage workflow home screen containing a representation of the patients' blood pressure based on the single blood pressure measurements of the patients. In this way, a user of the medical device 102 can perform triage on the series of unidentified patients as they arrive. As used in this document, a patient is an unidentified patient when the medical device 102 does not store information regarding the identity of the patient.

The monitoring workflow home screen is different than the non-monitoring workflow home screen. Further, as discussed below, the navigation options associated with the different workflows allows for efficient monitoring based on the environment in which the device is used. In various embodiments, the monitoring workflow home screen may be different than the non-monitoring workflow home screen in various ways. For example, in some embodiments, the monitoring workflow home screen includes at least one user-selectable control that is not included in the non-monitoring workflow home screen. In other embodiments, a representation of a physiological parameter in the monitoring workflow home screen has a different size than a representation of the same physiological parameter in the non-monitoring workflow home screen.

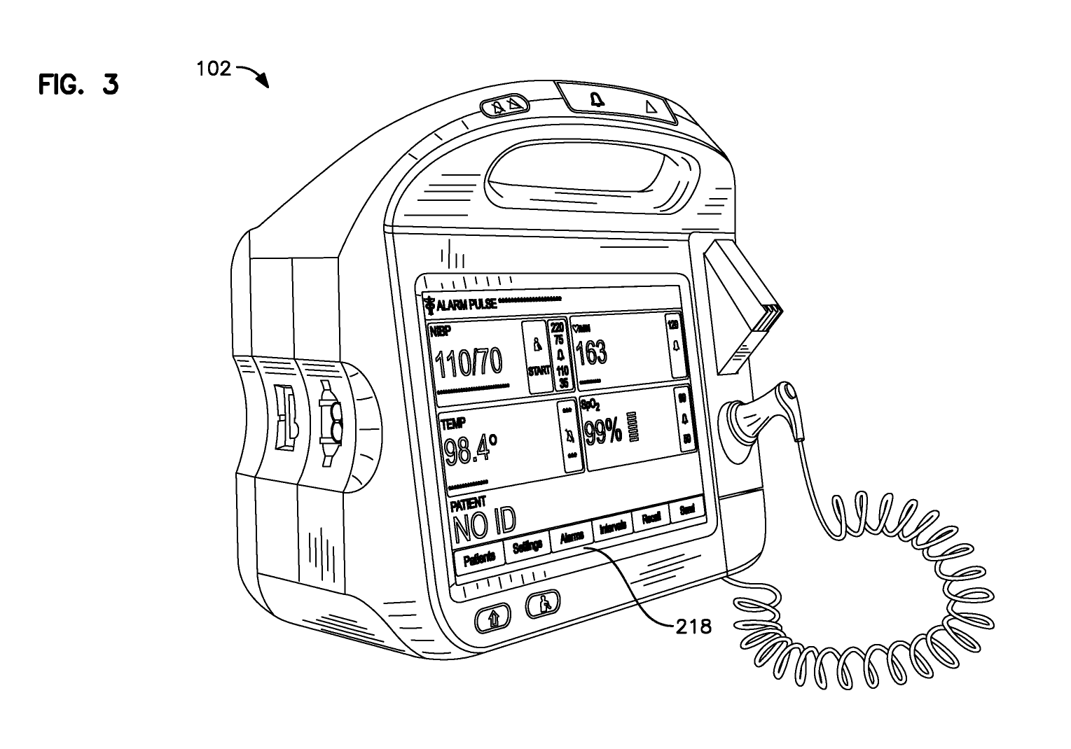

FIG. 3 illustrates an example user interface displayed on the display screen 218 of FIG. 2. The medical device 102 outputs and displays user interfaces discussed in this document on the display screen 218.

In some examples described herein, the physiological monitor device is a portable device. In other examples, the physiological monitor device is a non-portable device, such as a computing device like a workstation. Many configurations are possible.

The medical device 102 shown in FIGS. 2-3 is only one example of a medical device. All different types of medical devices used to collect patient data can be used.

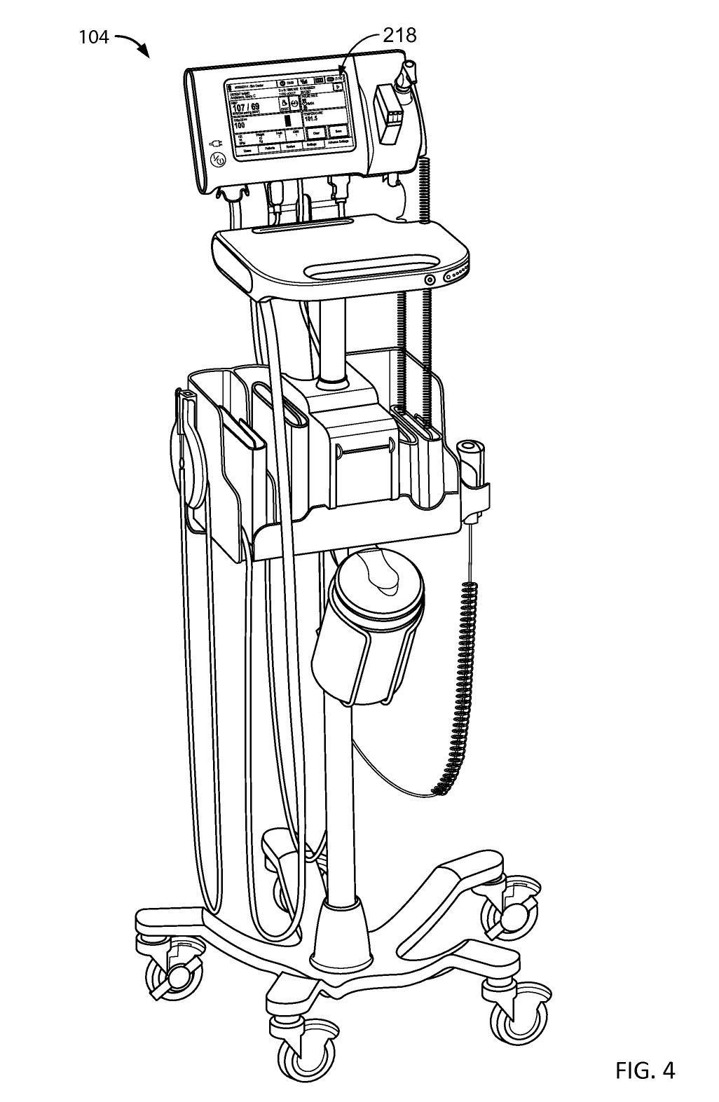

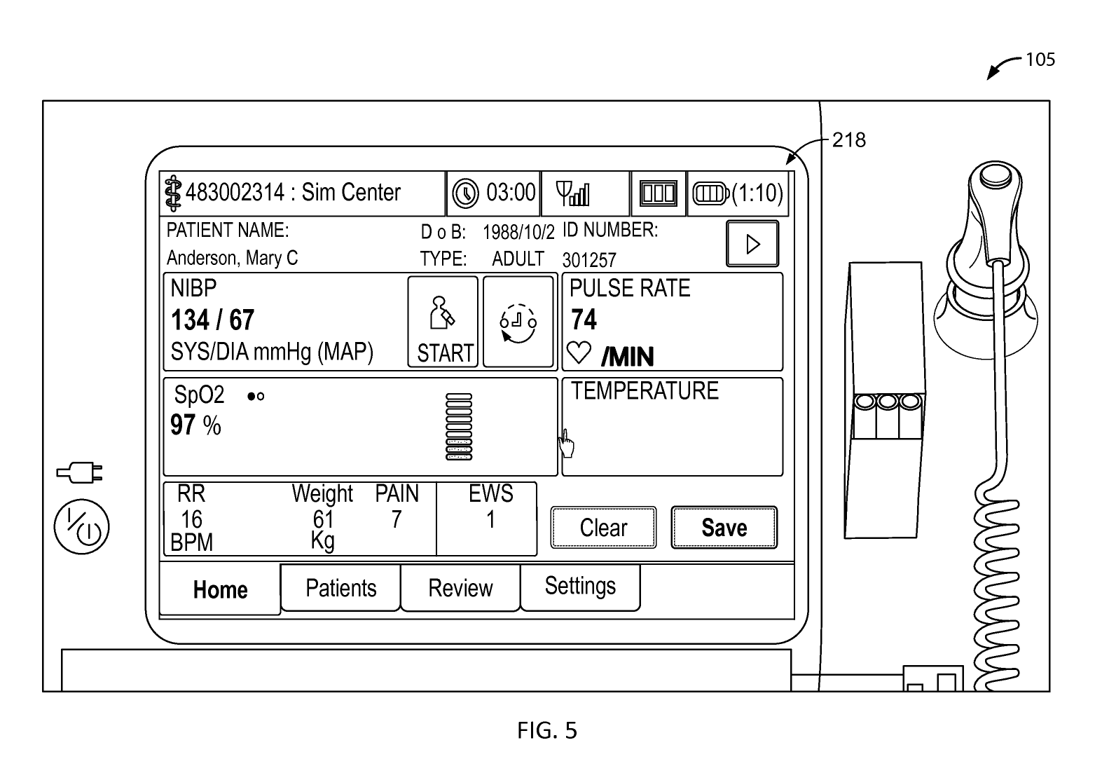

For example, the medical device 104 is shown in FIG. 4, and the medical device 105 is shown in FIG. 5. In this example, the medical devices 104, 105 are similar to the medical device 102 described above. The medical device 104 is shown on a mobile cart, and the medical device 104 is programmed in a manner similar to that described above to monitor physiological parameters of a patient. In some examples, the medical device 104 can be a more compact device that includes a touch screen (e.g., 7 inches) and the ability to execute multiple workflows. In embodiments, the medical device 105 is configured to be mounted on a wall and may also be programmed in a manner similar to that described above to monitor physiological parameters of a patient. Additionally, in some embodiments, the medical devices 104, 105 are stand-alone devices, which can mean that the medical devices 104, 105 are not part of a mobile cart or wall-mounted station.

The medical devices 102, 104 105 shown in FIGS. 2-5 are only examples of a medical device. In some examples described herein, the medical devices 102, 104, 105 are portable devices. In other examples, the medical devices 102, 104, 105 are non-portable devices, such as computing devices like workstations. All different types of medical devices used to collect patient data can be used. Many configurations are possible.

As further described below, one or more of the medical devices 102, 104, 105 can be used to automatically calculate and/or display an early warning score related to the current and/or historical state of the patient. As described above, some embodiments of the medical devices 102, 104, 105 operate in multiple workflows. Depending upon which workflow the medical devices 102, 104, 105 are operating in, the medical device 102, 104, 105 may operate to calculate and/or display an early warning score for a patient one or multiple times. For example, in a monitoring workflow, the medical devices 102, 104, 105 may operate to calculate (or update) the early warning score multiple times as the patient is being monitored. Alternatively, in a non-monitoring workflow, the medical devices 102, 104, 105 may operate to calculate the early warning score once after a patient's physiological measurements have been obtained.

Acute care teams have been using early warning scores for several years in an effort to provide more timely assessments and predictions to changes in patient acuity. SAPSII, APACHE, MEWS, PEWS, MEDS, REMS, ASSIST, and SCS are some of the many scoring systems that have been adopted in emergency department (ED), medical/surgical, general care, and ICU environments.

There have been recent nationwide movements to standardize scoring systems on general care floors in the UK and Australia using the National Early Warning Score (NEWS) and Between The Flags (BTF) protocols respectively.

The NEWS is based on a simple scoring system in which a score is allocated to physiological parameters that are already recorded for patients in general care settings. Six physiological parameters form the basis of the scoring system:
Respiratory rate
Oxygen saturation
Temperature
Systolic blood pressure
Pulse rate
Level of consciousness (AVPU)

Some of the parameters are measured by the medical devices 102, 104, 105. While others of the parameters are determined through observation of the patient. The observed parameters may be input into the medical devices 102, 104, 105 through a user interface. A score is allocated to each parameter as it is measured or input into the medical devices 102, 104, 105. The magnitude of the score reflects how extreme the parameter varies from the norm. The score is then aggregated and a patient's care plan is modified according to the protocol.

The Clinical Excellence Commission (CEC), a board-governed statutory health corporation located in New South Wales (NSW), developed and implemented Between the Flags (BTF). This program is being implemented across NSW.

BTF is an Early Warning Score program focused on early recognition of patient deterioration. This program builds on operational processes that are in place and is not intended to take clinical assessment away from the nurses. Nursing staff is expected to be able to have access to clinical data and to aggregate and analyze it to form a clinical assessment. To help with this effort, the program established standards outlining what observations should be recorded and what thresholds should trigger a response. Instead of a numbering system, the protocol focuses on human factors and the use of color.

These parameters form the basis of the scoring system:
Respiratory rate
Oxygen saturation
Temperature
Systolic blood pressure
Pulse rate

In example embodiments, the medical devices 102, 104, 105 are programmed to calculate early warning scores using one or more of the protocols described above. The devices 102, 104, 105 also are programmed to communicate those scores, both visually to the caregiver, as well as possibly to a central server, such as server device 112 to be stored in an electronic medical records (EMR) system. Further, the devices 102, 104, 105 are programmed to provide configurable alert messages based upon the calculated early warning scores.

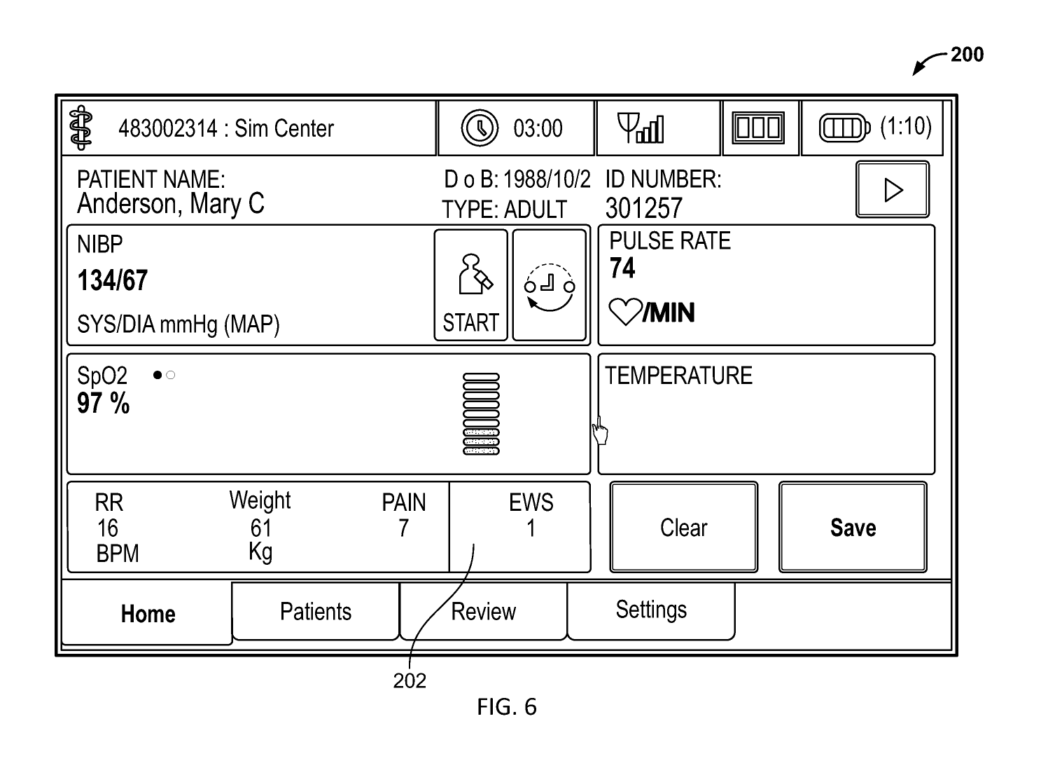

For example, referring now to FIG. 6, an example user interface 200 for one or more of the medical devices 102, 104, 105 is shown. This user interface 200 shows information about a plurality of the physiological parameters that are monitored by the devices 102, 104, 105. In addition, the interface 200 displays an early warning score 202 for the patient. In this example, the early warning score is a “1”.

This score can be configured by the hospital and/or caregiver to utilize one or more protocols to calculate the early warning score. Each parameter used to calculate the score can be configured, and the raw score shown on the interface 200 can be configured. For example, in this embodiment, the score is the average of the parameter scores used to calculate the score. So, if the parameter scores used to calculate the early warning score are 1, 3, 3, 1, the early warning score 202 would be shown as a “2”. In another example, each parameter score can be weighted differently.

In yet another example, the score shown on the interface 200 can simply be the highest of the parameter scores. So, if the parameter scores are 1, 3, 1, 2, then the interface 200 would show a “3” as the early warning score 202. In other examples, the early warning score can be an aggregate of the parameter scores.

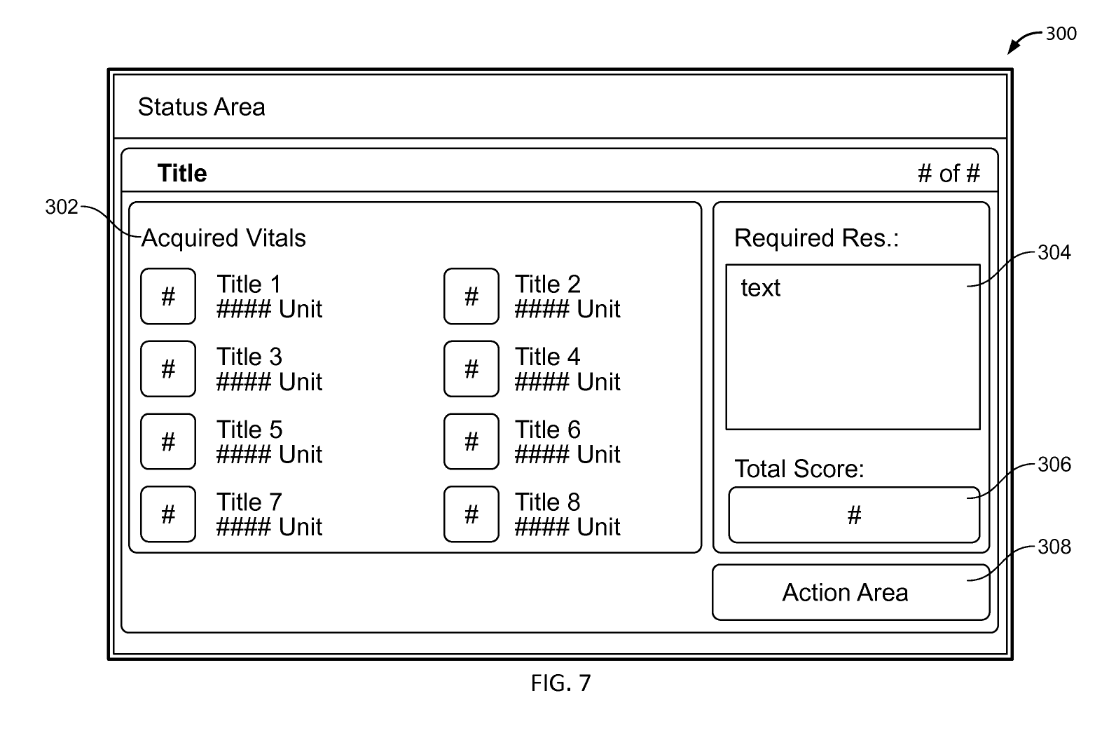

An interface 300 is shown in FIG. 7 that illustrates the various parameters that are used to calculate the early warning score, along with the total score and recommended action.

The interface 300 includes an area 302 in which up to 8 parameters that are used to calculate the early warning score 202 are shown. For each parameter, the title for the parameter can be provided, along with the proper units. For example, a title might be “Systolic BP,” and the units could be “mm Hg.” The required response field 304 is a text field that provides instructions to the caregiver should a given early warning score be displayed. For example, one such instruction could be “Check the patient for congestive heart failure.” Such an instruction can be provided in conjunction with one or more alarms, such as visual and/or audible alarms provided by the medical device 102, 104, 105.

The total score area 306 provides the calculated early warning score, such as the early warning score 202 described above. This can, for example, be an aggregate score, an average, and/or a high score of the various parameters used to calculate the early warning score.

Finally, the action area 308 allows the user to cycle through the various interfaces to input the desired early warning score parameters.

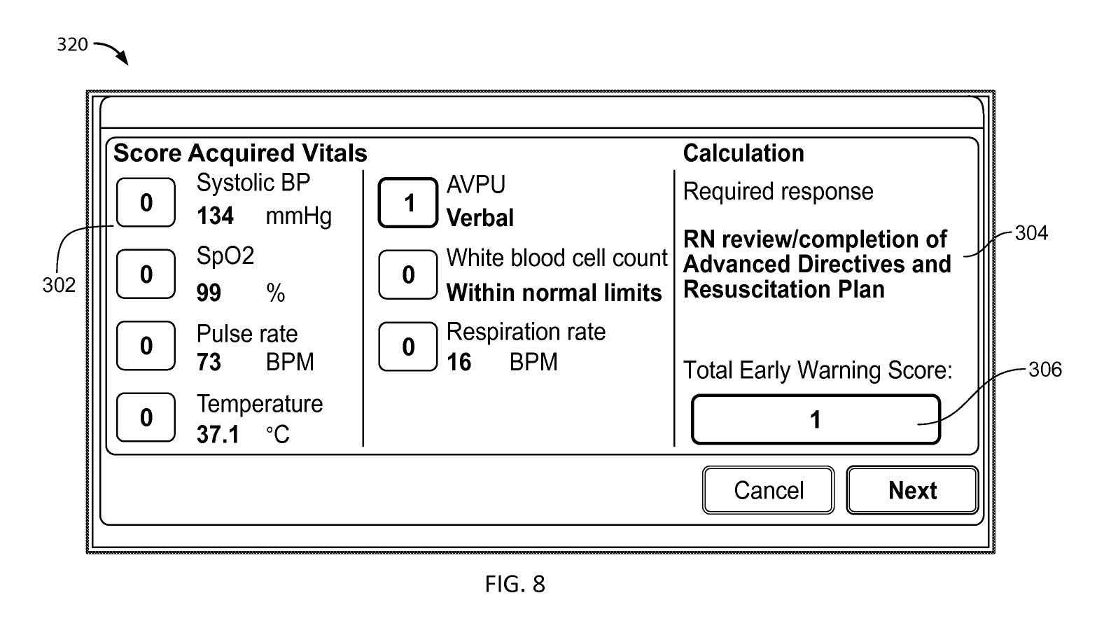

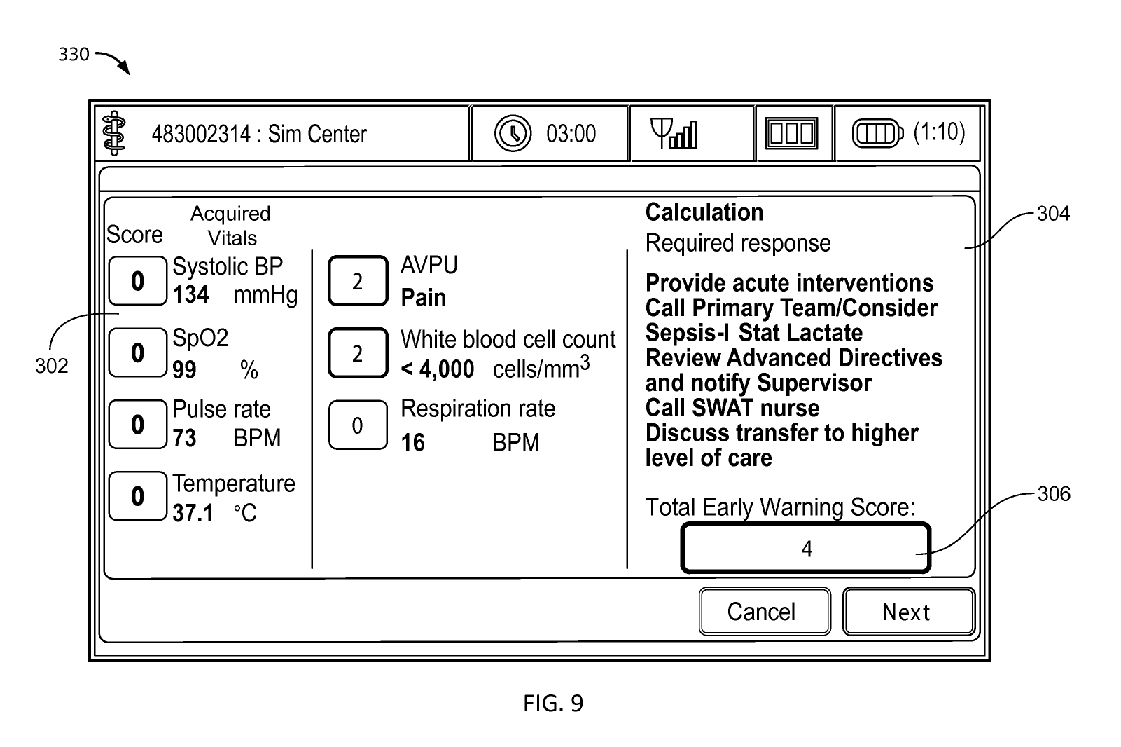

Examples of such interfaces are shown in FIGS. 8-9.

In FIG. 8, an example interface 320 shows the parameters used to calculate the early warning score, such as systolic BP, SpO2, pulse rate, temperature, AVPU, white blood cell count, and respiration rate. For each parameter, the score for that parameter is shown. For example, the score for systolic BP is “0”. The total score area 306 is a representation of the parameters shown, whether aggregate, average, or highest value. In the example given, the total score is “1” which is the highest score of the measured parameters. In this example, the early warning score is rated from 0-4, with 4 being the worst. Finally, the required response field 304 displays text giving instructions to the caregiver given the early warning score.

In FIG. 9, another example interface 330 shows similar information. The total score area 306 in this example in an aggregate of the parameters, and the required response field 304 provides different instructions to the caregiver based upon the score.

In some examples, the early warning score can be tailored to an individual or demographic so that even more precise information is provided. For example, the caregiver can select different early warning score protocols and/or configure those protocols given bibliographic and/or demographic information about the patient.

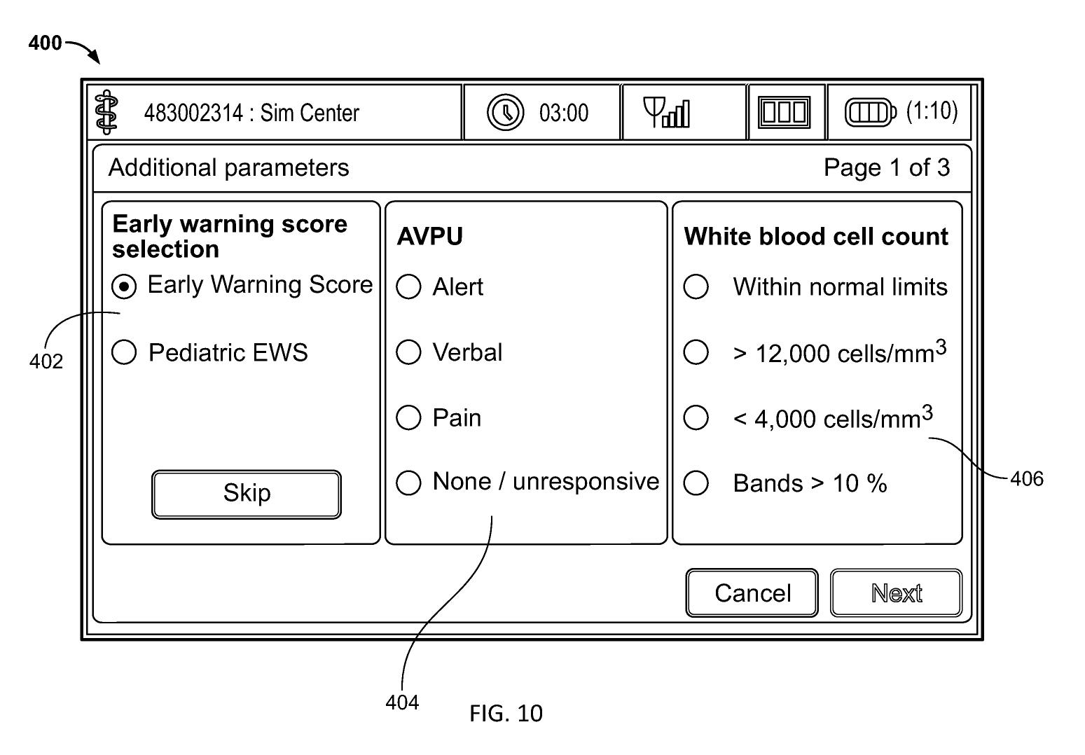

For example, referring now to FIG. 10, an example interface 400 for the medical devices 102, 104, 105 is shown. In this example, an area 402 allows the user to select between a standard early warning score protocol and a pediatric early warning score. This can be important because the scoring systems can differ significantly depending on demographic or other information about the patient, such as the patient's age.

In addition, for the selected protocol, the caregiver can select different parameters and how those parameters are weighted to calculate the early warning score. For example, in the area 404, the user can select a value for the level of consciousness (AVPU) parameter for the patient, and in area 406 the white blood cell count for the patient. By defining the values, the caregiver can modify the protocol to provide the desire warning.

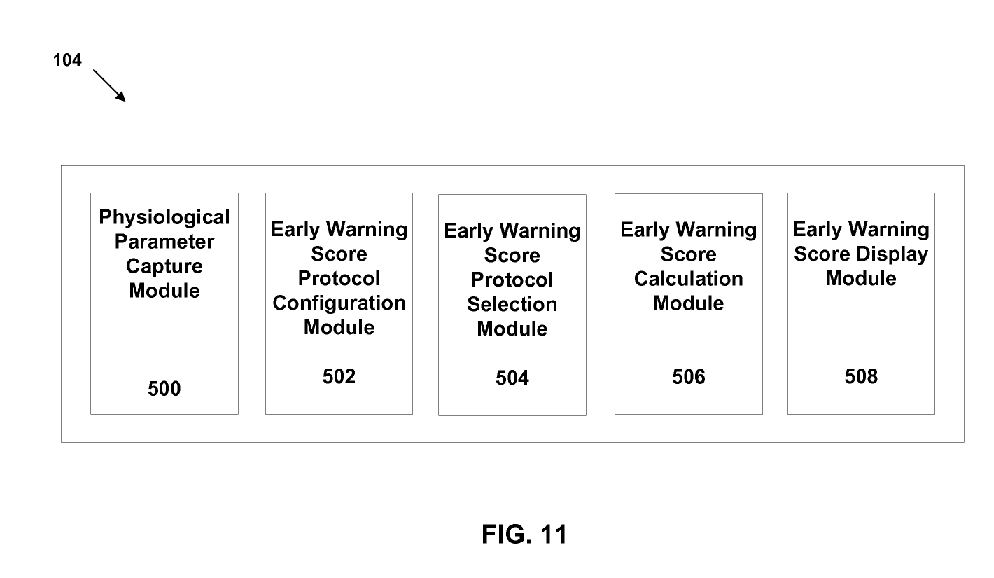

Referring now to FIG. 11, an example schematic of the medical devices 102, 104, 105 is shown in more detail. In this example, the medical devices 102, 104, 105 include a physiological parameter capture module 500, an early warning score protocol configuration module 502, an early warning score protocol selection module 504, an early warning score calculation module 506, and an early warning score display module 508.

The physiological parameter capture module 500 is a device that is configured to capture a physiological parameter of the patient. Some of the physiological parameters are captured directly by the physiological parameter capture module 500. For example, the physiological parameter capture module 500 can include one or more HCE modules to capture physiological parameters such as body temperature, SpO2, and NIBP. In some embodiments, the physiological parameter capture module 500 also includes a user interface that can be used by a caregiver to input physiological parameters that are observed, such as level of consciousness. Additionally, the physiological parameter capture module 500 can include an interface to receive physiological parameters over the network 108 such as from another medical device, a laboratory system, or an EMR system. White blood cell count is an example of a physiological parameter that can be received over the network 108. In other embodiments, other parameters are received over the network 108.

The early warning score protocol configuration module 502 operates to define and modify early warning score protocols. The early warning score protocol configuration module 502 can be used to modify an existing early warning score protocol, such as by replacing or adding one or more physiological parameters to the protocol, changing the mapping from values of physiological parameters to component scores, and changing the calculation method or formula used to combine the component scores. Additionally, the early warning score protocol configuration module 502 can be used to define new early warning score protocols. For example, the early warning score configuration module 502 can be used to enter a name of a protocol, select physiological parameters for the protocol, map values of those physiological parameters to component scores, and enter calculation methods for combining the component scores. Changes to existing early warning score protocols and newly defined early warning score protocols can be available locally, such as on one of the medical device 102, 104, 105, or globally, such as across the system 100. Additionally, in some embodiments, changes made using the early warning score protocol configuration module 502 may only be applied to a particular patient. Furthermore, the early warning score protocol configuration module 502 may accessible to all users of the medical devices 102, 104, 105, or alternatively, the early warning score protocol configuration module 502 is only accessible to administrators. Other embodiments are possible as well.

The early warning score protocol selection module 504 operates to select a particular early warning score protocol to apply to a particular patient or group of patients. For example, the early warning score protocol selection module 504 can be used to select an early warning score protocol based on the patient's age, such as a pediatric protocol for pediatric patients, a standard protocol for adult patients, and a geriatric protocol for geriatric patients. The early warning score protocol selection module 504 automatically selects or suggests an appropriate protocol for a patient based on demographic information about the patient. Alternatively, the early warning score protocol selection module 504 generates a user interface for a caregiver to select the appropriate protocol.

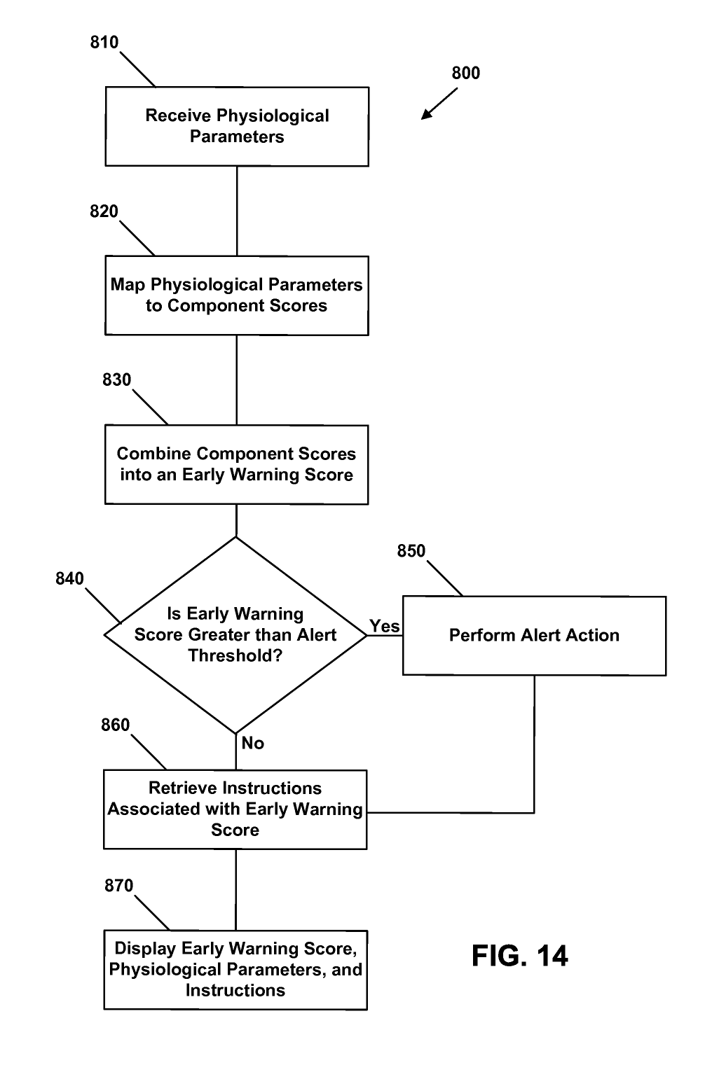

The early warning score calculation module 506 operates to calculate an early warning score according to the protocol selected by the early warning score protocol selection module 504. An example method of calculating the early warning score is illustrated and described in more detail with respect to FIG. 14.

The early warning score display module 508 operates to display the early warning score to a caregiver or other user of the medical device 102, 104, 105. The early warning score display module 508 generates a user interface to display the score. In some embodiments, the early warning score display module 508 also includes a visual indicator of the early warning score, such as a color or font. For example, an early warning score that indicates the patient is doing well may be displayed in green text using a plain font, while an early warning score that indicates that the patient is in need of additional care or monitoring may be displayed in red text using a bold font. Other embodiments are possible as well.

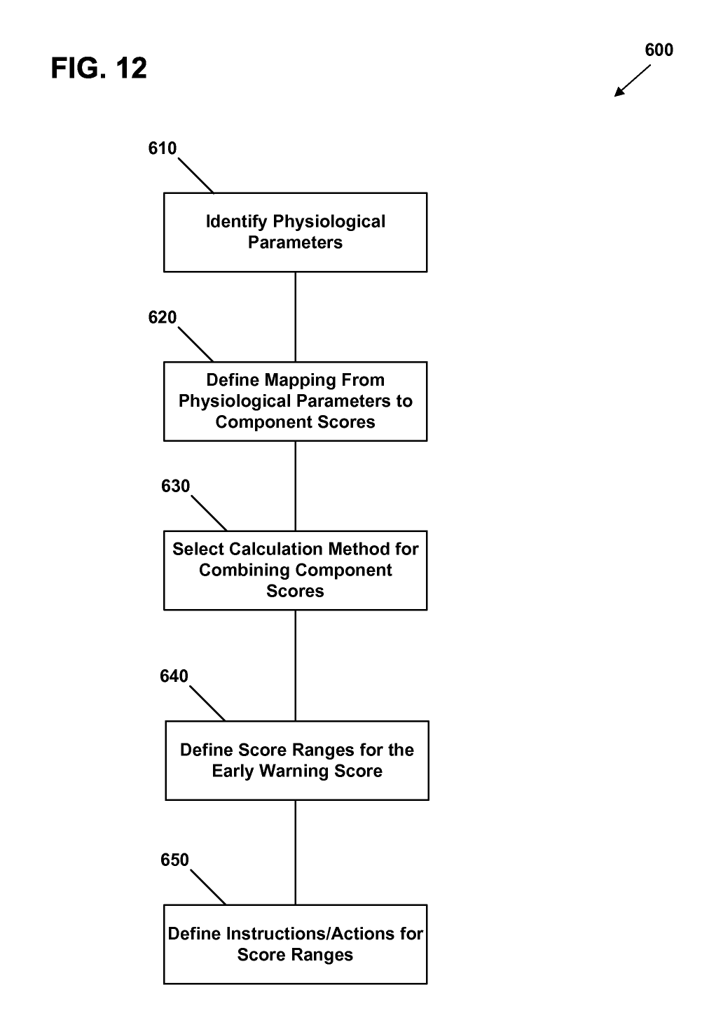

Referring now to FIG. 12, an example process 600 performed by some embodiments of the medical devices 102, 104, 105 is shown. The process operates to define an early warning score protocol.

At operation 610, physiological parameters are identified that are used by the early warning score. Example physiological parameters include body temperature, oxygen saturation, systolic blood pressure, diastolic blood pressure, mean arterial pressure, pulse rate, white blood cell count, and respiratory rate. Other embodiments use other physiological parameters as well. In some embodiments, between four and eight physiological parameters are identified for use to calculate an early warning score. However, in other embodiments, more or fewer physiological parameters are used to calculate the early warning score.

At operation 620, a mapping is defined from the physiological parameters to component scores that are used in calculating the early warning score. For some physiological parameters the mapping operates to convert a numeric measurement of a physiological parameter to a component score having an integer value from 0 to 3. The component score can represent the magnitude of the difference between the measured physiological parameter and a normal value, with a value of 0 representing normal and 3 representing extremely abnormal. For example, a systolic blood pressure measurement in the range of 100-130 mm Hg is assigned a value of 0; 80-100 mm Hg or 130-140 mm Hg is assigned a value of 1; 70-80 mm Hg or 140-150 mm Hg is assigned a value of 2; and less than 70 mm Hg or greater than 150 mm Hg is assigned a value of 3. In some embodiments, the value of systolic blood pressure is mapped differently. Further, other physiological parameters may be mapped differently. For example, with level of consciousness (AVPU), a score of A (awake) can be assigned the value 0; V (responsive to verbal stimulation) can be assigned the value 1; P (responsive to pain stimulation) can be assigned the value 2; and U (completely unresponsive) can be assigned the value 3. In other embodiments, other methods are used to assign component scores to various physiological parameters. For example, in some embodiments, a different range of component scores is used. Additionally, in some embodiments, the component score is represented as a decimal number rather than an integer. Accordingly, a mathematical formula may be used to map a measured physiological parameter to the component score.

At operation 630, a calculation method for combining the component scores into an early warning score is selected. Example calculation methods for combing the component scores include selecting the maximum component score, averaging the component scores, and summing the component scores. Additionally, in some embodiments, the component scores are weighted before being summed or averaged. In other embodiments, other calculation methods are used to combine the component scores into an early warning score.

At operation 640, ranges are defined for the early warning scores. The ranges can be used to define various instructions for a caregiver or actions to be taken by the medical devices 102, 104, 105 based on the early warning score. In some embodiments, the ranges may be equal to the possible integer values of the early warning scores. For example, in embodiments where the early warning score is an integer value, each of 0, 1, 2, and 3 can be defined as a separate range. The appropriate range values vary in different embodiments based on the mappings used in operation 620 and the calculation method for combining component scores used in operation 630.

At operation 650, instructions and actions are defined for the ranges for the early warning scores. The instructions are textual information for the caregiver. Alternatively, the instructions can include graphical information for the caregiver as well. The instructions can provide guidance for the caregiver about how to interpret the early warning score and what actions should be taken based on the early warning score. For example, the instructions can be based on policies at a health care facility. The actions, on the other hand, define steps for the medical device 102, 104, 105 to perform based on the early warning score. As an example, an early warning score in a range that indicates highly abnormal results can include the instructions “Transfer to Intensive Care Unit Immediately,” and trigger the action “sound alarm at monitoring station.” Other embodiments include different instructions and different actions.

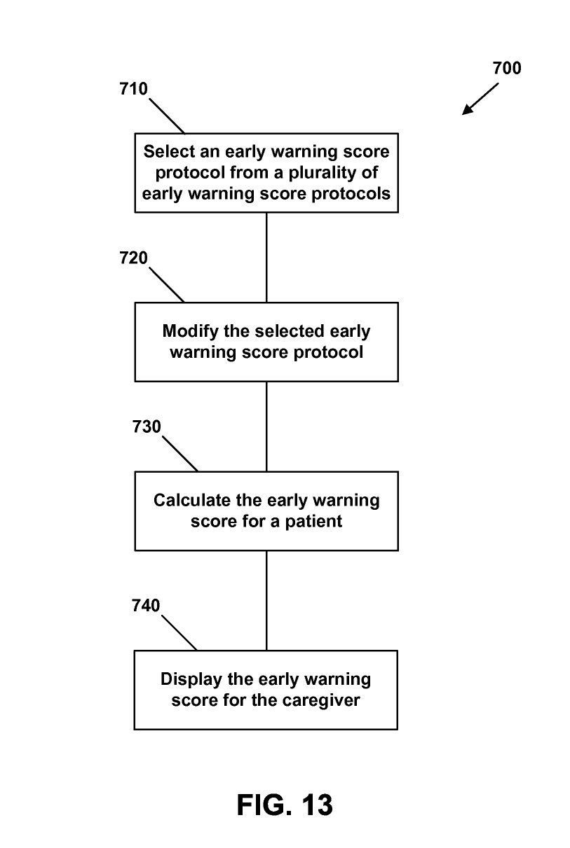

FIG. 13 shows an example process 700 performed by some embodiments of the medical device 102, 104, 105 to modify and use an early warning score protocol.

At operation 710, an early warning score protocol is selected from a plurality of early warning score protocols. The early warning score protocol can be selected by a caregiver using a user interface of the medical devices 102, 104, 105. Alternatively, the early warning score protocol can be selected automatically by the medical device 102, 104, 105 based on a demographic or biographic information about the patient such as age, gender, weight, medical history, etc.

At operation 720, the selected early warning score protocol is modified. The early warning score protocol can be modified to include additional or different physiological parameters. Additionally, the early warning score protocol can be modified to map physiological parameters to component scores differently or to combine the component scores differently to calculate the early warning score. However, in some embodiments, the early warning score protocol is not modified.

At operation 730, the early warning score is calculated for a patient. The early warning score can be calculated based on physiological parameters that have recently been recorded. In some embodiments, the early warning score is automatically recalculated when a physiological parameters is recorded or updated. Further, in some embodiments, the early warning score is calculated only when each of the physiological parameters upon which the early warning score is based have been recorded within a predetermined time period, such as fifteen minutes, one hour, four hours, or one day. Other embodiments use other time periods. In this manner, the early warning score may be more accurate, timely, and relevant.

At operation 740, the early warning score is displayed for the caregiver. The early warning score can be displayed on a user interface of the medical devices 102, 104, 105. Additionally, instructions for the caregiver that are based on the early warning score can be shown as well. Further, in some embodiments, the recorded physiological parameters or component scores that are used in calculating the early warning score are also shown. The user interface of some embodiments displays abnormal early warning scores, physiological parameters, or component scores differently than normal early warning scores, physiological parameters, or component scores. In this manner, the caregiver's attention can be drawn to patients in need of more care, monitoring, or attention and to particular abnormal physiological parameters.

FIG. 14 shows another example process 800 performed by some embodiments of the medical devices 102, 104, 105 to calculate and display an early warning score according to an early warning score protocol. The early waning score may be calculated using a predefined early warning score protocol or a customized early warning score protocol.

At operation 810, physiological parameters are received. The physiological parameters may be measured by the medical devices 102, 104, 105, input by a caregiver, or received over the network 108. At operation 820, the physiological parameters are mapped to component scores. The mapping operates according to the early warning score protocol and serves to assign a value that is associated with the magnitude of abnormality of the recorded physiological parameters. At operation 830, the component scores are combined to calculate an early warning score according to the early warning score protocol. The component scores can be combined using various methods such as those described previously with respect to operation 630.

At operation 840, the early warning score is compared to an alert threshold. If the early warning score is greater than the alert threshold, the process continues to operation 850 where an alert action is performed. Both the alert threshold and the alert action can be defined in the early warning score protocol. Examples of alert actions include sounding an audible alarm, flashing a light, sending a message over the network 108, or placing a call or sending a text message to a phone. In this manner, the medical devices 102, 104, 105 can draw the caregiver's attention to a patient who may need additional care or monitoring. Other embodiments include other alert action as well. Additionally, some embodiments do not include alert thresholds or alert actions.

At operation 860, instructions associated with the early warning score can be retrieved. The instructions may be stored in memory, in one or more database tables, or in one or more text or other types of files. Other embodiments are possible as well. At operation 870, the early warning score and the instructions are displayed for the caregiver.

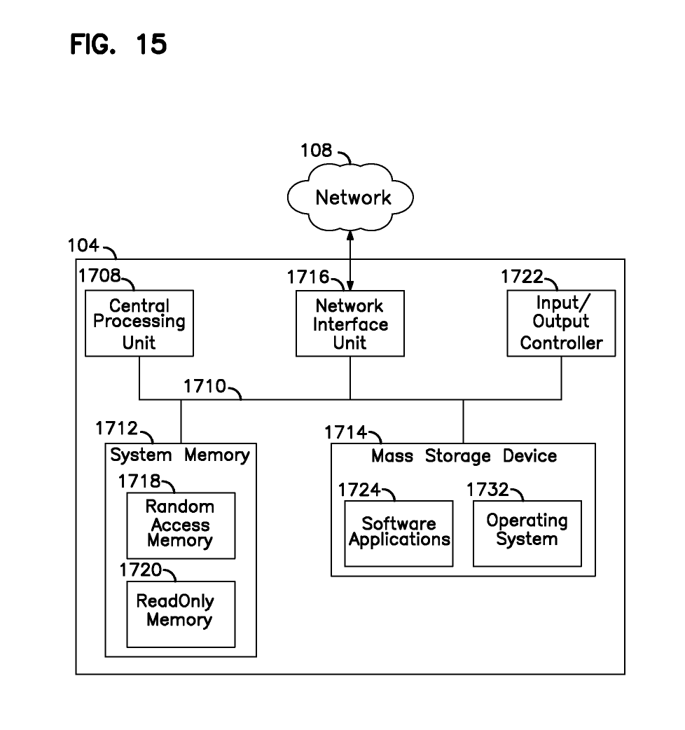

FIG. 15 illustrates example physical components of a computing device, such as the medical devices 102, 104, 105. As illustrated, the device includes at least one central processing unit (“CPU”) 1708, a system memory 1712, and a system bus 1710 that couples the system memory 1712 to the CPU 1708. The system memory 1712 includes a random access memory (“RAM”) 1718 and a read-only memory (“ROM”) 1720. A basic input/output system containing the basic routines that help to transfer information between elements within the device, such as during startup, is stored in the ROM 1720. The device further includes a mass storage device 1714. The mass storage device 1714 is able to store software instructions and data. The central processing unit 1708 is an example of a processing device.

The mass storage device 1714 is connected to the CPU 1708 through a mass storage controller (not shown) connected to the bus 1710. The mass storage device 1714 and its associated computer-readable data storage media provide non-volatile, non-transitory storage for the device. Although the description of computer-readable data storage media contained herein refers to a mass storage device, such as a hard disk or CD-ROM drive, it should be appreciated by those skilled in the art that computer-readable data storage media can be any available non-transitory, physical device or article of manufacture from which the device can read data and/or instructions. The mass storage device 1714 is an example of a computer-readable storage device.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the device.

According to various embodiments of the invention, the device may operate in a networked environment using logical connections to remote network devices through the network 108, such as a local network, the Internet, or another type of network. The device connects to the network 108 through a network interface unit 1716 connected to the bus 1710. The network interface unit 1716 may also be utilized to connect to other types of networks and remote computing systems. The device also includes an input/output controller 1722 for receiving and processing input from a number of other devices, including a keyboard, a mouse, a touch user interface display screen, or another type of input device. Similarly, the input/output controller 1722 may provide output to a touch user interface display screen, a printer, or other type of output device.

As mentioned above, the mass storage device 1714 and the RAM 1718 of the device can store software instructions and data. The software instructions include an operating system 1732 suitable for controlling the operation of the device. The mass storage device 1714 and/or the RAM 1718 also store software instructions, that when executed by the CPU 1708, cause the device to provide the functionality of the device discussed in this document. For example, the mass storage device 1714 and/or the RAM 1718 can store software instructions that, when executed by the CPU 1708, cause the physiological monitor device to display the interfaces 200, 300, 330, and 400, various home screens, and other screens.

Although the example medical devices described herein are devices used to monitor patients, other types of medical devices can also be used. For example, the different components of the CONNEX™ system, such as the intermediary servers that communication with the monitoring devices, can also require maintenance in the form of firmware and software updates. These intermediary servers can be managed by the systems and methods described herein to update the maintenance requirements of the servers.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A method for providing an early warning system for a patient, the method comprising:
allowing a caregiver to select among a plurality of early warning score protocols;
allowing the caregiver to modify the selected early warning score protocol;
automatically calculating, by a medical device, an early warning score for the patient; and
displaying the early warning score for the caregiver.

2. The method of claim 1, wherein at least one of the early warning score protocols is based on at least one physiological measurement of the patient captured by the medical device.

3. The method of claim 2, wherein the at least one physiological measurement is selected from a group of physiological measurement types comprising:
body temperature;
oxygen saturation;
systolic blood pressure;
diastolic blood pressure;
mean arterial pressure;
pulse rate;
white blood cell count; and
respiratory rate.

4. The method of claim 2, further comprising:
displaying the at least one physiological measurement that the early warning score is based on.

5. The method of claim 1, wherein at least one of the early warning score protocols is based on at least one physiological observation of the patient.

6. The method of claim 5, wherein the physiological observation is level of consciousness and a value for level of consciousness is input by a caregiver using a user interface of the medical device.

7. The method of claim 1, wherein at least one of the early warning score protocols is based on a physiological parameter received over a network.

8. The method of claim 1, wherein the plurality of selected early warning score protocols includes at least one of:
SAPSII;
APACHE;
MEWS;
PEWS;
MEDS;
REMS;
BTF;
ASSIST; and
SCS.

9. The method of claim 1, wherein the selected early warning score protocol is selected based on demographic information associated with the patient.

10. The method of claim 9, wherein the demographic information includes at least one of age and gender.

11. The method of claim 1, wherein the early warning score is calculated by mapping a plurality of physiological parameters to component scores and combining the component scores, wherein the component scores are based on the magnitude of variance from a normal value.

12. The method of claim 11, wherein the component scores are combined by at least one of: averaging the component scores, selecting the maximum value from the component scores, and summing the component scores.

13. The method of claim 1, further comprising displaying instructions for the caregiver, the instructions being selected based on the early warning score.

14. The method of claim 1, further comprising displaying a visual indicator based on the early warning score, the visual indicator being associated with a magnitude of abnormality of the early warning score.

15. The method of claim 1, further comprising automatically recalculating the early warning score when a new value for a physiological parameter is received.

16. A method for defining an early warning score protocol for a patient, the method comprising:
identifying a plurality of physiological parameters;
defining a mapping from the plurality of physiological parameters to a plurality of component score;
defining a calculation method for combining the plurality of component scores into an early warning score;
specifying a plurality of ranges for the early warning score; and
inputting instructions for at least one of the plurality of ranges.

17. The method of claim 16, further comprising:
specifying an alert threshold based on the early warning score; and
specifying an alert action to be performed when the early warning score exceeds the alert threshold.

18. The method of claim 17, wherein the alert action comprises generating an audible alarm.

19. The method of claim 16, wherein the mapping for at least one of the plurality of physiological parameters assigns an integer value to a range of values measured for the physiological parameter.

20. A medical device comprising:
at least one health care equipment module configured to measure at least one physiological parameter of a patient;
a display screen;
a computing device, the computing device comprising:
at least one processing device; and
at least one computer readable data storage device storing instructions that, when executed by the at least one processing device, cause the medical device to:
allow a caregiver to select among a plurality of early warning score protocols, wherein at least one of the early warning score protocols is targeted to specific patients based on demographic information;
allow the caregiver to modify the selected early warning score protocol;
automatically calculate an early warning score for the patient using a physiological parameter captured by the at least one health care equipment module; and
display a user interface on the display screen, the user interface including the early warning score and instructions for the caregiver that are selected based on the early warning score.
